(19) United States
(12) Patent Application Publication
(10) Pub. No.: US 2005/0261210 A1
(43) Pub. Date: Nov. 24, 2005

(54) Nonsense suppressor agents in treatment of cutaneous and gastrointestinal disorders

(76) Inventors: Rajiv S. Bhatnagar, Pacifica, CA (US); Steven E. Brenner, Berkeley, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/054,155

(22) Filed: Feb. 8, 2005

Related U.S. Application Data
(60) Provisional application No. 60/546,485, filed on Feb. 19, 2004.

(51) Int. Cl. A61K 31/704

(52) U.S. Cl. 514/35; 514/37; 514/38; 514/39

(57) ABSTRACT

The invention features methods of treating a inherited or acquired cutaneous or gastrointestinal (GI) disorder caused by or having a symptom caused by a nonsense mutation by local administration of a nonsense suppressor agent (e.g., an aminoglycoside or nucleic acid encoding a suppressor tRNA) to the skin or intraluminal GI surface so as to provide for phenotypic suppression of the nonsense mutation.

### CROSS-REFERENCE TO RELATED APPLICATION

This application claims the benefit of U.S. provisional application Ser. No. 60/546,485, filed Feb. 19, 2004, which application is incorporated herein by reference in its entirety.

### GOVERNMENT RIGHTS

This invention was made with government support under federal grant no. K22HG00056 awarded by the National Institutes of Health. The United States Government may have certain rights in this invention.

### FIELD OF THE INVENTION

The present invention generally relates to treatment of disorders caused by nonsense mutations, particularly cutaneous or gastrointestinal disorders.

### BACKGROUND OF THE INVENTION

Genetic diseases result from alterations in the sequence of the human genome (mutations), resulting in alteration of normal physiologic processes. Disease-associated mutations can be of several forms: deletions of sequences, insertions of extraneous sequence into a gene, and point mutations that alter a single nucleotide of the genomic sequence. Point mutations within the coding sequence of a gene may induce no change in the encoded protein sequence (synonymous mutations), alter one amino acid to another (missense mutation), or change an amino acid coding mutation to a termination codon (nonsense mutation).

While missense mutations often result in subtle alterations in protein structure and function, most nonsense mutations are predicted to result in a loss of production of protein from the affected gene, due to nonsense-mediated mRNA decay, a cellular editing mechanism that prevents the expression of prematurely terminated proteins. As such, nonsense mutations often have a phenotype equivalent to deletion of the affected allele. In cases in which nonsense-mediated mRNA decay does not occur, nonsense mutations result in the synthesis of truncated proteins that may have gains or losses of function relative to the full-length protein.

Genetic diseases caused by point mutations can be inherited or acquired. Acquired point mutations can result from, for example, exposure to radiation (e.g., solar, galactic, man-made) or chemicals. Acquired mutations can be an underlying cause of many cancers (Cleaver et al. Front Biosci, 2002. 7:d1024-43).

Aminoglycoside antibiotics mediate their antibacterial activity by binding of the drug to the decoding site of the 16S subunit of the prokaryotic ribosome (Woodcock, et al. EMBO J., 1991. 10(10):3099-3103), and disrupt codon-anticodon recognition during protein translation, with a resultant loss of fidelity in protein synthesis. This results in introduction of missense amino acids and readthrough of termination codons (Edelmann et al. Cell, 1977. 10(1):131-7).

Aminoglycosides can similarly affect translation in eukaryotic cells. The decoding site in eukaryotic ribosomes is structurally similar to the prokaryotic counterpart, and aminoglycosides can bind, with reduced affinity, to eukaryotic 18S rRNA (Recht et al. EMBO J, 1999. 18(11):3133-8). Several studies have demonstrated that aminoglycosides can induce misreading during translation in eukaryotic systems (Wilhelm et al. Biochemistry, 1978. 17(7): 1149-53). In particular, the ability of aminoglycosides to promote readthrough of termination codons in eukaryotic translation has been studied in some detail (Burke et al. Nucleic Acids Res, 1985. 13(17):6265-72). The efficiency with which readthrough occurs depends on the sequence of the stop codon, neighboring nucleic acid sequence, and the structure of the aminoglycoside (Howard et al. Ann Neurol, 2000. 48(2):164-9; Keeling et al. J Mol Med, 2002. 80(6):367-76).

The ability of aminoglycosides to suppress translational termination has led to trials of their use in treatment of genetic diseases caused by nonsense mutations. Muscular dystrophy and cystic fibrosis are the best studied examples.

Muscular dystrophy is caused by mutations in the human dystrophin gene. The gene encodes a protein with an important role as part of a multiprotein complex in muscle cell membranes. The most severe muscular dystrophy phenotypes arise from nonsense mutations or frameshifts that introduce premature termination codons (Kerr et al. Hum Genet, 2001. 109(4):402-7). The mdx mouse, an animal model of the severe Duchenne form of muscular dystrophy, has a nonsense mutation in its dystrophin gene (Sicinski et al. Science, 1989. 244(4912):1578-80.) No dystrophin protein is detectable in muscle cells from mdx mice, due to probable nonsense-mediated decay of the mRNA bearing the premature termination codon. Treatment of cultured muscle cells from mdx mice with gentamicin resulted in detectable levels of dystrophin (Barton-Davis et al. J Clin Invest, 1999. 104(4):375-81). Subsequent systemic treatment of mdx mice with gentamicin resulted in expression of the protein throughout the animal, with resultant protection against the muscle injury observed in untreated mdx mice (Barton-Davis et al. 1999, supra). Extension of these studies to patients with Duchenne muscular dystrophy caused by nonsense mutations have demonstrated treatment-dependent expression of dystrophin in some patients (Politano et al. Acta Myol, 2003. 22(1):15-21), and a lack of response in others (Wagner et al. Ann Neurol, 2001. 49(6):706-11).

Cystic fibrosis results from mutations in the CFTR gene, encoding a regulated chloride channel important for the function of respiratory and digestive tract epithelial cells. Like the dystrophinopathies, cystic fibrosis is associated with many different mutations within the underlying gene. Nonsense mutations in the CFTR are associated with severe phenotypes. Gentamicin and other aminoglycoside antibiotics have been demonstrated to restore expression of functional CFTR protein in cultured respiratory epithelial cells harboring nonsense mutations of the CFTR gene (Bedwell et al. Nat Med, 1997. 3(11): 1280-4), and in a transgenic mouse model of nonsense-mutated CFTR (Du et al. J Mol Med, 2002. 80(9):595-604).

A placebo-controlled trial demonstrated the effectiveness of gentamicin in cystic fibrosis patients with nonsense mutation in the CFTR gene (Wilschanski et al. N Engl J Med, 2003. 349(15):1433-41). In this trial application of gentamicin to the nasal mucosa resulted in restoration of functional CFTR protein. The study demonstrated the ability of this aminoglycoside to correct the molecular defect underlying the fatal genetic disease. However, the bulk of the affected tissues in patients with cystic fibrosis are not accessible to treatment by application to the nasal mucosa.

Unfortunately, systemic administration of aminoglycoside antibiotics is limited by nephrotoxic and ototoxic effects (Kahlmeter et al. J Antimicrob Chemother, 1984. 13 Suppl A:9-22). This limits the use of aminoglycosides in the treatment of many diseases.

Several cutaneous and gastrointestinal disorders are associated with a nonsense mutation in a gene. Examples of cutaneous disorders associated with nonsense mutations include basal cell nevus syndrome (PTCH gene), sporadic basal cell carcinoma (PTCH gene), melanoma (CDKN2a gene), junctional epidermolysis bullosa (LAMB3, LAMC2, LAMA3 genes), generalized atrophic benign epidermolysis bullosa (COL17A1 gene), dystrophic epidermolysis bullosa (COL7A1 gene), Hailey-Hailey disease (ATP2C1 gene), Darier's disease (ATP2A2 gene), lamellar icthyosis (TGM1 gene), X-linked icthyosis (STS gene), xeroderma pigmentosa (XPA, XPC, XPG genes), Bloom syndrome (BLM gene), striate palmo-plantar keratoderma (DSP, DSG1 genes), Cockayne syndrome (ERCC6 gene), oculocutaneous albinism (TYR, TYRP1 genes), Hermansky-Pudlack syndrome (HPS1, HPS4 genes), ataxia-telangiectasia (ATM gene), Griscelli syndrome (RAB27A, MYO5A genes), and ectodermal dysplasia/skin fragility (PKP1 gene).

Conventional therapy for these cutaneous and gastrointestinal disorders does not address the underlying genetic defects that lead to the disorder phenotype. For example, many of the cutaneous disorders above are simply treated so as to mitigate symptoms of the disease (e.g., treatment of inflammation and discomfort by administration of corticosteroids, treatment of pain, and the like). Several of the disorders have no treatment, but rather focus on prevention of complications that result from having the disorder (e.g., avoiding sunlight where the patient has xeroderma pigmentosa).

The present invention addresses the need in the field for therapies for nonsense mutation-associated cutaneous and GI disorders.

Literature

The following references, as well as those cited above, may be of interest:
U.S. Publication Nos. US 20020123470; and US 20010051607. U.S. Pat. Nos. 5,840,702; and 6,475,993.
Suppressor tRNAs: Beier et al. Nucleic Acids Res, 2001. 29(23):4767-82; Atkinson et al. Nucleic Acids Res, 1994. 22(8):1327-34; and Buvoli et al. Mol Cell Biol. May 2000;20(9):3116-24); and U.S. Pat. No. 6,309,830
Delivery of nucleic acid to skin: U.S. Pat. No. 6,197,755; U.S. Pat. No. 6,654,636; U.S. Pat. No. 6,673,776; U.S. Pat. No. 6,087,341; and U.S. Pat. No. 5,589,466.

Wilschanski et al. N Engl J Med 2003. 349(15):1433-41; Wilschanski et al. Am J Respir Crit Care Med. March 2000;161(3 Pt 1):860-5; Helip-Wooley et al. Mol Genet Metab. February 2000;75(2):128-33).
Arakawa et al. J Biochem (Tokyo). November 2003;134(5):751-8; Thompson et al. J Mol Biol. Sep. 13, 2000;322(2):273-9.

### SUMMARY OF THE INVENTION

The invention features methods of treating a inherited or acquired cutaneous or gastrointestinal (GI) disorder caused by or having a symptom caused by a nonsense mutation by localized administration of a nonsense suppressor agent (e.g., an aminoglycoside or nucleic acid encoding a suppressor tRNA) to the skin or intraluminal GI surface so as to provide for phenotypic suppression of the nonsense mutation.

One advantage of the present invention is that localized administration to the affected tissue suppresses the genetic basis of the disease without exposure of unaffected tissues to agents. This is particularly advantageous where the agent if can have adverse side effects if administered systemically. For example, topical administration of an aminoglycoside to the skin to treat a cutaneous disorder avoids delivery of the aminoglycoside to the systemic circulation at a dose that is associated with toxicity. Similarly, orally administered aminoglycosides are poorly absorbed into the circulation, but can achieve high concentrations within the gastrointestinal lumen for treatment of GI disorders.

Another advantage of the invention is that the methods can be applied to treatment of inherited or acquired diseases associated with nonsense mutations.

Before the present invention is described in more detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention.

The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a nonsense mutation” includes a plurality of such nonsense mutations and reference to “the aminoglycoside” includes reference to one or more such aminoglycosides and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

Overview

The invention is based on the use of a nonsense suppressor agent to treat cutaneous or gastrointestinal (GI) disorders associated with a nonsense mutation.

Nonsense suppressor agents can be of two general classes. A first class of agents includes compounds that disrupt codon-anticodon recognition during protein translation in a eukaryotic cell, so as to promote readthrough of a nonsense codon. These agents can act by, for example, binding to a ribosome so as to affect its activity in initiating translation or promoting polypeptide chain elongation, or both. For example, nonsense suppressor agents of this class can act by binding to rRNA (e.g., by reducing binding affinity to 18S rRNA). These agents are generally referred to herein as “small molecule nonsense suppressor agents”. Examples of such nonsense suppressor agents include aminoglycosides chloramphenicol, oxazolidinones, and derivatives or analogs thereof that retain activity in promoting readthrough of a nonsense mutation.

Reference to aminoglycosides as exemplary of this first general class of nonsense suppressor agents is not meant to be limiting, but rather only exemplary of such agents suitable for use in the present invention.

A second class of agents are those that provide the eukaryotic translational machinery with a tRNA that provides for incorporation of an amino acid in a polypeptide where the mRNA normally encodes a stop codon, e.g., suppressor tRNAs. An example of such a nonsense suppressor agent is a nucleic acid encoding a suppressor tRNA. Reference to aminoglycosides as exemplary of this second general class of nonsense suppressor agents is not meant to be limiting, but rather only exemplary of such agents suitable for use in the present invention.

Administration of nonsense suppressor agents is generally local (i.e., not systemic) so as to avoid delivery of levels of agents associated with toxicity to the systemic circulation. Local delivery provides a therapeutic local concentration of where needed to treat cutaneous or GI diseases, without exposing unaffected tissues to unnecessary medication.

In one embodiment local delivery is by topical administration to the skin or intraluminal GI surface. This delivery route is particularly advantageous where the nonsense suppressor agent is an aminoglycoside. Topical application of aminoglycosides takes advantage of the benefits of the mutation-directed therapy while reducing the systemic toxicity associated with intravenous, intramuscular, subcutaneous or other systemic route of administration of these agents.

The invention provides a medical treatment for a number of diseases of the skin and GI disorders. Numerous hereditary diseases, and some acquired diseases, are caused by point mutations that change a codon from encoding an amino acid to one encoding protein termination (nonsense mutations). These mutations result in loss or alteration of the function of the protein encoded by the gene in question. Nonsense suppressor agents, such as aminoglycosides and suppressor tRNAs, can cause readthrough of termination codons, such that the full-length protein encoded by the mutated gene can be expressed. Thus, the genetic defect underlying the disease can be circumvented by this technology.

Definitions

A “nonsense mutation” is a mutation in a nucleic acid sequence that causes premature termination of translation of an messenger RNA into a polypeptide by altering a codon that encodes an amino acid to a sequence encoding a translational termination.

By “readthrough” is meant translation of the sequence of an mRNA without regard to a stop codon present in the sequence so that translation continues 3′ of the stop codon.

By “stop codon” is meant a codon that is at least part of a genetic signal for translation to end. In mammalian cells, these codons are UAG, UAA, UGA (corresponding to the DNA sequences TAG, TAA, and TGA, respectively).

By “termination signal” is meant a stop codon that signals for the end of translation of an mRNA. Sequences flanking (e.g., 3′ of the stop codon) can be part of a termination signal, as in a tetranucleotide termination signal. The tetranucleotide termination signal (the stop codon and the nucleotide 3′ of the stop codon) can be a primary determinant for aminoglycoside-mediated nonsense mutations suppression. (Keeling et al. J Mol Med. June 2002;80(6):367-76. Epub Jan. 25, 2002).

A “nonsense suppressor agent” is an agent that facilitates phenotypic suppression of a nonsense mutation in a gene when administered to a cell. Aminoglycosides and nucleic acid encoding a suppressor tRNA are exemplary nonsense suppressor agents of particular interest.

A “suppressor tRNA” is a tRNA which provides for incorporation of an amino acid in a polypeptide where the mRNA normally encodes a stop codon.

A “cutaneous disorder” refers to a disorder of the skin, particularly disorders of the epidermis or dermis, more particularly the epidermis, components of the skin. “Epidermis” includes: the stratum corneum, stratum lucidum, stratum granulosum, stratum spinosum, and stratum germinativum (stratum basale, basal cell layer). Of particular interest are cutaneous disorders that are associated with a nonsense mutation(s).

A “gastrointestinal disorder” or “GI disorder” refers to a disorder of the GI tract, including the mouth, pharynx, esophagus, stomach, and duodenum (e.g., small intestine, large intestine (e.g., colon)). Of particular interest are GI disorders that are associated with a nonsense mutation(s).

By a “disorder associated with a nonsense mutation” is meant a disorder that is caused by a nonsense mutation, and/or having one ore more symptoms caused by a nonsense mutation, where the nonsense mutation prevents production of a full-length gene product in an affected cell of the subject. “Disorders associated with a nonsense mutation” encompasses disorders in which a single gene contains one or more nonsense mutations as well as disorders in which two or more (multiple) genes contain one or more nonsense mutations.

An “affected individual” (or an “affected patient” or “affected subject”) is an individual who is diagnosed as having a particular disorder or is suspected of having such a disorder due to presentation of disorder-associated symptoms (e.g., a cutaneous disorder of GI disorder associated with a nonsense mutation). A “non-affected individual” is an individual who is not diagnosed as having a particular disorder or not suspected of having such a disorder. Cutaneous and GI disorders associated with a nonsense mutation(s) and methods of diagnosing such disorders are known in the art.

“Development” or “progression” of a disorder as used herein means initial manifestations and/or ensuing progression of the disorder. Development of a disorder can be detectable and assessed using standard clinical techniques, which are selected according to the particular disorder for which diagnosis is desired. Development also refers to disease progression that may be undetectable. For purposes of this invention, development or progression refers to the biological course of the disease state. “Development” includes occurrence, recurrence, and onset. As used herein “onset” or “occurrence” of a disorder includes initial onset and/or recurrence.

As used herein, “delaying development” of a disorder means to defer, hinder, slow, retard, stabilize, and/or postpone development of the disease. This delay can be of varying lengths of time, depending on the history of the disorder and/or the medical profile of the individual being treated. As is evident to one skilled in the art, a sufficient or significant delay can, in effect, encompass prevention, in that the individual does not develop detectable disease. A method that “delays” development of disease is a method that reduces the extent of the disease in a given time frame, when compared to not using the method. Such comparisons are typically based on clinical studies, using a statistically significant number of subjects, although this knowledge can be based upon anecdotal evidence. “Delaying development” can mean that the extent and/or undesirable clinical manifestations are lessened and/or time course of the progression is slowed or lengthened, as compared to not administering the agent. Thus the term also includes, but is not limited to, alleviation of symptoms, diminishment of extent of disease, stabilized (i.e., not worsening) state of disease, delay or slowing of disease progression, and remission (whether partial or total) whether detectable or undetectable.

As used herein, “biological sample” encompasses a variety of sample types obtained from an individual and can be used in a diagnostic or monitoring assay. The definition encompasses any appropriate sample that may be obtained from a subject, including blood and other liquid samples of biological origin; and solid tissue samples such as a biopsy specimen or tissue cultures or cells derived therefrom, and the progeny thereof. The definition also includes samples that have been manipulated in any way after their procurement, such as by treatment with reagents, solubilization, or enrichment for certain components, such as proteins or polynucleotides. The term “biological sample” encompasses a clinical sample, and also includes cells in culture, cell supernatants, cell lysates, serum, plasma, biological fluid, and tissue samples. Generally, the sample will be, or be derived from, peripheral blood and as such is a “blood sample”. In some cases, the blood will have been enriched for a macrophage fraction, by using, for example, glass or plastic adherence.

A “blood sample” is a biological sample which is derived from blood, preferably peripheral (or circulating) blood. A blood sample may be, for example, whole blood, plasma or serum.

As used herein, an “effective amount” (e.g., of an agent) is an amount (of the agent) that produces a desired and/or beneficial result. An effective amount can be administered in one or more administrations. In some embodiments, an effective amount is an amount sufficient to provide for increased production of a functional gene product encoded in a gene containing one or more nonsense mutations, which mutation(s) in the absence of therapy does not provide for adequate expression of a functional gene production (e.g., in the absence of therapy according to the invention, expression of the nonsense mutation-containing gene results in production of a truncated gene product). An “amount effective to stimulate expression of a functional gene product” or an “amount effective to stimulate expression of a full-length gene product” is an amount that provides for production of a gene product encoded by a gene contained one or more nonsense mutations so as to provide for an increase in functional gene product so as to provide for phenotypic suppression of an associated cutaneous or GI disorder. In some embodiments, expression of a functional gene product can be increased according to the invention by at least about 25%, preferably at least about 50%, more preferably at least about 75%, and even more preferably at least about 90%. An increase in expression of a functional gene product can have desirable concomitant effects, such as to palliate, ameliorate, stabilize, reverse, slow or delay progression of disease, delay and/or even prevent onset of disease.

“Phenotypic suppression” of a disorder is meant to refer to increase production of a functional gene product encoded by a gene containing one or more nonsense mutations so as to provide for a phenotype in a cell that is similar to that of an unaffected cell (e.g., a cell in which the same gene does not contain the nonsense mutation(s)).

“Treatment” or “treating” as used herein means any therapeutic intervention in a subject, usually a mammalian subject, generally a human subject, including: (i) prevention, that is, causing overt clinical symptoms not to develop, e.g., preventing disease progression to a harmful state; (ii) inhibition, that is, arresting the development or further development of clinical symptoms, e.g., mitigating existing clinical symptoms; and/or (iii) relief, that is, causing the regression of clinical symptoms, e.g., causing relief from clinical symptoms.

An “individual” is a vertebrate, preferably a mammal, more preferably a human. Mammals include, but are not limited to, farm animals, sport animals, rodents, primates, and pets.

The terms “subject” and “patient” mean a member or members of any mammalian or non-mammalian species that may have a need for the pharmaceutical methods, compositions and treatments described herein. Subjects and patients thus include, without limitation, primate (including humans), canine, feline, ungulate (e.g., equine, bovine, swine (e.g., pig)), avian, and other subjects. Humans and non-human animals having commercial importance (e.g., livestock and domesticated animals) are of particular interest.

“Mammal” means a member or members of any mammalian species, and includes, by way of example, canines; felines; equines; bovines; ovines; rodentia, etc. and primates, particularly humans. Non-human animal models, particularly mammals, e.g. primate, murine, lagomorpha, etc. may be used for experimental investigations.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable excipient (e.g., pharmaceutically acceptable diluent, carrier or vehicle).

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. “A pharmaceutically acceptable excipient” as used in the specification and claims includes both one and more than one such excipient.

Nonsense Suppressor Agents

Nonsense suppressor agents useful in the methods of the invention include agents that, when administered to the skin or GI tract as described herein, facilitate phenotypic suppression of a nonsense mutation in an affected cell. Exemplary nonsense suppressor agents include, but are not necessarily limited to aminoglycosides, chloramphenicol, oxazolidinones, and deriviatives or analogs thereof that retain activity in promoting readthrough. Aminoglycosides are of particular interest. Further nonsense suppressor agents of interest include nucleic acid encoding suppressor tRNAs.

Small Molecule Nonsense Suppressor Agents and Formulations

Small molecule nonsense suppressor agents include any compound that can disrupt codon-anticodon recognition in a eukaryotic cell so as to promote readthrough of a nonsense mutation. Examples of such small molecule nonsense suppressor agents include aminoglycosides, chloramphenicol, oxazolidinones, and derivatives or analogs thereof that retain activity in promoting readthrough of a nonsense mutation.

Aminoglycosides are a group of broad-spectrum antibiotics which were initially isolated from species of Streptomyces or Micomonospora fungi. “Aminoglycosides” generally refers to a compound having an amino sugar, and amino-or guanido-substituted inositol ring which are attached by a glycosidic linkage to a hexose nucleus resulting in a polycationic and highly polar compound, or derivatives or analogs thereof, which compounds suppress nonsense mutations in a cell, particularly in a mammalian cell. Without being held to theory, aminoglycosides suppress nonsense mutations by affecting the translational machinery in cells (e.g., the ribosome or particularly ribosomal subunits, e.g., by binding to eukaryotic 18S rRNA) so to cause “readthrough” of a stop codon of the nonsense mutation rather than termination of polypeptide chain synthesis. In some embodiments, aminoglycosides of interest are those that suppress nonsense mutations in a context-dependent manner in a mammalian translation system, e.g., aminoglycosides for which the tetranucleotide termination signal (the stop codon and the nucleotide 3′ of the stop codon) is a primary determinant for aminoglycoside-mediated suppression.

“Aminoglycosides” in the context of the invention include naturally-occurring compounds and derivatives or analogs thereof, as well as synthetically produced compounds and derivatives thereof. Exemplary aminoglycosides include, but are not limited to, gentamicin, streptomycin, amikacin, kanamycin, tobramycin, netilmicin, neomycin, framycetin, negamycin, paromomycin, sisomicin, G-418, and derivatives or analogs thereof. In some embodiments, amikacin, gentamicin, negamycin, and analogs or derivatives thereof are of particular interest.

“Oxazolidinones” in the context of the invention include naturally-occurring compounds and derivatives or analogs thereof, as well as synthetically produced compounds and derivatives thereof. Exemplary oxazolidinones include, but are not limited to, linezolid, eperzolid, and analogs or derivatives and analogs thereof.

Small molecule nonsense suppressor agents such as aminoglycosides can be selected according to, for example, the site and route of administration, the relative toxicities, the gene having the nonsense mutation to be suppressed, and the levels of termination suppression (“readthrough”) desired. For example, levels of termination suppression by gentamicin and amikacin are higher than those provided by tobramycin in an in vitro assay, while amikacin produces higher level of read-through than gentamicin in some contexts. (see, e.g., Keeling et al. J Mol Med. June 2002;80(6):367-76. Epub Jan. 25, 2002).

Small molecule nonsense suppressor agents, such as aminoglycosides, can be administered in a free base or free acid form (that is, as the free compound and not as a salt), or as a pharmaceutically acceptable salt(s) of the compound(s) can also be used. Pharmaceutically acceptable salts are those salts which retain the biological activity of the free compounds and which are not biologically or otherwise undesirable. As appropriate, stereoisomers of the compounds disclosed can also be used in the invention, including diastereomers and enantiomers, as well as mixtures of stereoisomers, including, but not limited to, racemic mixtures. Unless stereochemistry is explicitly indicated in a structure, the structure is intended to embrace all possible stereoisomers of the compound depicted.

Nucleic Acid Encoding Nonsense suppressor tRNAs

Nucleic acid encoding a suppressor tRNA for expression in a mammalian cell, and methods of production of such nucleic acid, have been described (see, e.g., Beier et al. Nucleic Acids Res, 2001. 29(23):4767-82; Atkinson et al. Nucleic Acids Res, 1994. 22(8):1327-34; and Buvoli et al. Mol Cell Biol. May 2002;20(9):3116-24). In vivo use of suppressor tRNA in a mouse model of nonsense mutation gene therapy has been described (see, e.g., Buvoli et al. 2000, supra).

For example, U.S. Pat. No. 6,309,830 describes exemplary suppressor tRNAs which can find use in the methods of the present invention. Based upon the knowledge of known human tRNA sequences, synthetic oligonucleotides relating to opal, amber, or ochre mutations are constructed. Briefly, an oligonucleotide is synthesized which comprises the structural component of a known tRNA gene. The sequence of this oligonucleotide is designed based upon the known sequence with substitutions made in the anticodon region of the tRNA causing the specific tRNA to recognize a nonsense or any other specific or desired mutation. For example, the sequence of human serine tRNA having an anticodon of TCG can be modified to include a substitution of TCA the complement of the opal mutation to cause the tRNA to recognize the opal stop codon rather than the traditional serine codon.

Nucleic acids encoding tRNAs can be designed to contain only the structural sequence encoding the tRNA molecule as well as a small portion (around 20 nt)of the 3′ flanking region. The 5′ region is omitted to result in a nucleic acid molecule (e.g., oligonucleotide) that is small and easy to handle (e.g., around 100 nucleotides in length). The suppressor tRNA-encoding nucleic acid sequence comprises the structural component of the gene and includes about 15 bases from the 3′ flanking region and none of the 5′ noncoding region. Suppressor tRNA-encoding nucleic acid can be isolated (e.g., from a recombinant cell) or synthetically synthesized using standard molecular biology and biochemical techniques.

Suppressor tRNA-encoding nucleic acid can be provided in a variety of forms for administration. For example, the nucleic acid can be provided in a virus (e.g., replication defective retrovirus, adenovirus, or adeno-associated virus, and the like), in connection with a permeation enhancer to provide for penetration of the nucleic acid into the skin and into skin cells, as a cationic lipid formulation, or other suitable formulation.

Administration and Formulations

An aminoglycoside for use in the methods of the invention can be provided in any suitable formulation, which can be selected according to the desired route of administration, the disorder to be treated, and route of administration. Formulations of aminoglycosides, and corresponding routes of administration, of particular interest in the invention include those suitable for administration to the skin or to a GI surface.

Nonsense suppressor agent formulations are generally dosed in vivo corresponding to the body weight of the subject. Normally the agent is administered at regular intervals. Those of skill in the art will readily appreciate that actual dosages and regimen will vary as a function of the nonsense suppressor agent, formulation, the severity of the symptoms, the susceptibility of the subject to treatment and/or side effects, and the like. Dosages are readily and routinely determinable by those of skill in the art by a variety of means.

The regimen of administration (e.g., dose combined with frequency of administration) will generally involve administration in an amount and at a frequency to provide for a desired effect, e.g., administration of an amount effective to provide for improvement in one or more symptoms of the cutaneous or GI disorder being treated. For example, a nonsense suppressor agent can be administered for 2, 3, 4, 5, 6, 7, 8, 9, 10 or more consecutive days, which administration period may be reinitiated after 1, 2, 3 or more weeks following the last dose. In general, a goal of therapy according to the invention is to restore sufficient expression of the functional product of the mutated gene associated with the cutaneous or GI disorder so as to treat the disease phenotype.

Kits with unit doses of nonsense suppressor agents (e.g., aminoglycosides), usually in suitable dosage forms ready for administration, are provided. In such kits, in addition to the containers containing the unit doses will be an informational package insert describing the use and attendant benefits of nonsense suppressor agents (such as an aminoglycoside or nucleic acid encoding a suppressor tRNA) in treating cutaneous or GI disorders according to the invention, particularly by local delivery, especially topical delivery.

Treatment of Cutaneous Disorders

Where the disorder to be treated is a cutaneous disorder, nonsense suppressor agent (e.g., an aminoglycoside or nucleic acid encoding a suppressor tRNA) is formulated for administration to the skin. Such formulations include, for example, topical, intradermal, transdermal, subdermal, or subcutaneous formulations, or other formulations. Agents can be administered as a bolus, or through sustained release administration (including controlled release) and the like. Additional agents and therapies can be administered in combination, either in the same of different formulations, to the extent such are compatible with the methods of the invention.

The nonsense suppressor agents can be administered alone or in various combinations. Where administered in combination, nonsense suppressor agents can be administered in conjunction with other agents. Of particular interest are agents that promote penetration of the nonsense suppressor agent so as to improve its bioavailability for acting upon the relevant cells. Also of particular interest are agents for administration in conjunction with nonsense suppressor agents are those agents suitable for protective, palliative or supportive care of the subject. The phrase “in conjunction with” means that an agent is administered prior to, concurrently, or after other substance or therapy. Combination therapy thus encompasses administration of agents in the same or separate formulations, and administration at the same or different times. Examples of agents for administration in conjunction with an agent include, but are not limited to topical retinoids (e.g. tretinoin, tazarotene, adapalene), cyclooxygenase inhibitors (e.g. diclofenac), penetration enhancers (e.g., keratolytic agents (e.g glycolic acid, salicylic acid)), and the like.

In one embodiment of particular interest, administration of the agent is by local administration, e.g., delivery of the agent to the skin in a manner so as to avoid significant systemic delivery of the agent, e.g., by administering the agent in a manner so that the agent is not delivered to the vascular bed of the skin (e.g., nonvascular delivery). Where the agent is an aminoglycoside, local administration is of particular interest so as to avoid delivery of the aminoglycoside to the systemic circulation at levels that may be associated with undesirable side effects (e.g., toxicity, including nephrotoxicity and ototoxicity).

In one embodiment, the agent is an aminoglycoside which is locally administered as a topical formulation suitable for administration to the skin of the subject. Exemplary topical formulations of aminoglycosides are commercially available, e.g., gentamicin sulfate is available as a topical cream, ointment, or solution (0.1-0.3% base) (see, e.g., FDA application nos. 062307, 062531, 062427, 062351, 062533, 064093, 064163, 062635, and 062477); and neomycin sulfate (formulated in conjunction with one or more of hydrocortisone, hydrocortisone acetate, polymyxin B sulfate, and bacitracin-zinc (see, e.g., FDA application nos. 050168 and 050218).

It should be noted that formulations having higher concentrations of an aminoglycoside are also suitable for use in the methods of the invention, since local administration (e.g., topical administration) is not associated with the toxicity that is associated with systemic administration (e.g., by intravenous administration). Such aminoglycoside formulations can be, for example, about 2, 3, 4, 5, 6, 7, 8, 9 or 10 times higher than aminoglycoside concentrations of conventional intravenous formulations.

Where the agent is a nucleic acid encoding a suppressor tRNA, delivery to the skin can be accomplished according to methods known in the art. See, e.g., U.S. Pat. No. 6,197,755 (urea and nucleic acid for topical delivery of nucleic acid to skin); U.S. Pat. No. 6,654,636 (delivery of nucleic acid into skin by pulsed electrical field); U.S. Pat. No. 6,673,776 (nucleic acid delivery to skin); U.S. Pat. No. 6,087,341 (introduction of nucleic acid into skin cells by topical application); and U.S. Pat. No. 5,589,466 (injection into skin or transdermal).

Treatment of a cutaneous disorder according to the invention can be accomplished by transient or long-term phenotypic suppression. For example, phenotypic suppression by administration of a nucleic acid encoding a tRNA can result from transient expression of the nucleic acid in the cell without incorporation of the nucleic acid into the host cell genome. The duration of phenotypic suppression can also be affected by the turnover of cells from the epidermal surface (e.g., natural sloughing of cells over time from the subject).

Doses and dosage regimen can be readily determined by the ordinarily skilled clinical practitioner, will generally involve administration in an amount and at a frequency to provide for a desired effect, e.g., administration of an amount effective to provide for improvement in one or more symptoms of the cutaneous disorder. Effective doses of topical aminoglycosides will depend both on the phenotype of the condition being treated, as it relates to stratum corneum barrier function; and on the sensitivity of the individual mutation to the therapeutic effects of the agent.

Initial treatment of a condition that results in disruption of the stratum corneum would reasonably use of an about 0.1% formulation of gentamicin applied, for example, twice daily. More frequent application, application with occlusion, application in combination with an appropriate penetration enhancer, the use of higher concentration of the agent (including the commercially available 0.3% preparations, or more concentrated preparations up to 10%), or combinations thereof can be used for conditions with normal or enhanced barrier function, or if a therapeutic response is not observed with the initial dosing regimen. It is noted that higher concentrations of the agent can be administered, with the proviso that the regimen is adjusted so as to provide the desired effect and limit toxicity (e.g., avoid systemic toxicity). Exemplary signs of toxicity or onset of toxicity can include, for example, irritation, allergy, and the like. Thus one goal of the methods of the invention is to provide for a beneficial effect (e.g., in suppressing a nonsense mutation so as to provide complete or at least partial phenotypic suppression so as to at least mitigate symptoms of the disorder being treated) while at the same time minimizing toxicity and/or symptom of toxicity.

Similar considerations will guide the dosing of other aminoglycosides, other compounds, and related agents. Where the agent used is a nucleic acid encoding a suppressor tRNA, the dosing regimen will vary according to the method used to accomplish delivery, as described above.

Treatment of GI Disorders

A GI disorder amenable to treatment according to the invention includes, for example, GI cancers that result from one or more nonsense mutations, e.g., in a tumor suppressor gene (e.g., p53), which cancers includes sporadic and familial colon cancer.

Where the disorder to be treated is a GI disorder, nonsense suppressor agent (e.g., an aminoglycoside or nucleic acid encoding a suppressor tRNA) is formulated for administration to local delivery to an intraluminal surface of the GI tract, which is also referred to herein as topical administration. Topical formulations are those suitable for administration to GI mucosa that is present on the intraluminal surface. Topical delivery normally involves administration to an intraluminal surface of the gastrointestinal tract, e.g., the mouth, naso-pharynx, pharynx, esophagus, stomach, small intestine, and large intestine, or the vaginal or anorectal mucosa. Topical administration can be indirect (e.g., through administration of the agent by a route that with time provides for contact between the agent and the intraluminal GI surface) or direct (e.g., by placing the agent formulation directly onto the intraluminal GI surface).

Formulations suitable for local delivery to the GI tract include, for example, enteral formulations (e.g., oral, rectal, and the like). Agents can be administered as a bolus, or through sustained release administration (including controlled release) and the like. Additional agents and therapies can be administered in combination, either in the same of different formulations, to the extent such are compatible with the methods of the invention.

The nonsense suppressor agents can be administered alone or in various combinations. Where administered in combination, nonsense suppressor agents can be administered in conjunction with other agents, particularly those suitable for protective, palliative or supportive care of the subject. The phrase “in conjunction with” means that an agent is administered prior to, concurrently, or after other substance or therapy. Examples of agents for administration in conjunction with an agent include, but are not limited to cyclooxygenase inhibitors (e.g. aspirin, sulindac, celecoxib), folic acid, calcium, and the like. Chemoprophylactic compounds are of particular interest for use in combination therapy.

In one embodiment of the method of treatment of a nonsense mutation-associated GI disorder, the nonsense suppressor agent is an aminoglycoside. In this embodiment, the aminoglycoside is administered so as to provide for localized delivery of the aminoglycoside, while preferably avoiding delivery of the aminoglycoside to the systemic circulation at levels associated with toxicity (e.g., nephrotoxicity and ototoxicity).

Administration and formulations suitable for this embodiment include enteral routes of administration. Because bioavailability of aminoglycosides through oral delivery is generally poor (due to the highly polar nature of the aminoglycoside compound), localized delivery of an aminoglycoside to an intraluminal surface of the GI tract can be accomplished by oral administration.

Doses and dosage regimen can be readily determined by the ordinarily skilled clinical practitioner, will generally involve administration in an amount and at a frequency to provide for a desired effect, e.g., administration of an amount effective to provide for improvement in one or more symptoms of the GI disorder.

Generally, oral administration of aminoglycoside in the treatment of a GI disorder will involve a dose that is not sufficient for systemic delivery of an antimicrobially (e.g., antibacterially) effective dose. For example, the aminoglycoside can be delivered to the gastrointestinal lumen in a dose that results in less systemic absorption than that effective or required for antimicrobial therapy. For example, for treatment of colonic conditions, paromomycin can initially be administered orally at a dose of about 35-45 mg/kg per day, divided in three doses.

Exemplary oral formulations of aminoglycosides are commercially available, e.g., neomycin sulfate is available as an oral tablet or solution (see, e.g., FDA application nos. 065010 and 060304); kanamycin sulfate is available as an oral capsule (see, e.g., FDA application no 062726); paromomycin is available as an oral capsule (see, e.g., FDA application nos. 064171, 062310).

Other routes of administration suitable in this embodiment include administration of the aminoglycoside as a suppository or enema, or formulated in a viscous fluid for treatment of esophageal conditions, with total doses similar to the oral administration outlined above.

In another embodiment, the nonsense suppressor agent is a nucleic acid encoding a tRNA, which tRNA is for phenotypic suppression of a nonsense mutation present in a gene in an affected cell of the GI tract. Methods for administration of a nucleic acid for expression in a GI cell are known in the art (see, e.g., U.S. Pat. Nos. 6,258,789; 6,677,313 (using nucleic acid loaded polymeric microparticles); U.S. Pat. No. 6,656,498 (describing use of cationic liposomes)).

Treatment of the GI disorder according to the invention can be accomplished by transient or long-term phenotypic suppression. For example, phenotypic suppression by administration of a nucleic acid encoding a tRNA can result from transient expression of the nucleic acid in the cell without incorporation of the nucleic acid into the host cell genome. The duration of phenotypic suppression can also be affected by the turnover of cells from the GI intraluminal surface (e.g., natural sloughing of cells over time in the subject).

Subjects Amendable to Treatment and Monitoring of Therapy

Cutaneous disorders or gastrointestinal (GI) disorders, caused by a nonsense mutation, and/or having one ore more symptoms caused by a nonsense mutation, are amenable to therapy according to the invention. In general, cutaneous and GI disorders amenably to therapy are those in which a significant phenotype is caused by the altered expression of one or more genes harboring at least one nonsense mutation.

In the context of a cutaneous disorder, the nonsense mutation is generally present in a gene in a cell of the epidermis or in the most superficial aspect of the dermis. In the context of a GI disorder, the nonsense mutation is generally present in a gene in a cell of the epithelial lining of the intraluminal surface of the GI tract. These tissues are accessible for topical therapy.

In one embodiment, individuals suitable for therapy involving administration of a nonsense suppressor agent (e.g., an aminoglycoside) according to the invention include individuals who have been diagnosed as having, “afflicted with” (e.g., diagnosed as having, suffering from and/or displaying one or more clinical symptoms of such a disorder), or who have been adjudged to be at high risk for developing a nonsense mutation-associated cutaneous or GI disorder. An “at risk” or “high risk” individual is an individual who has a discrete and significant risk of developing a nonsense mutation-associated cutaneous or GI disorder.

An “at risk” or “high risk” individual may or may not have detectable disease, and may or may not have displayed detectable disease prior to receiving the therapy according to the method(s) described herein. “High risk” (or “at risk”) denotes that an individual has one or more risk factors, which are measurable parameters that correlate with development of disease. An individual having one or more of these risk factors has a higher probability of developing disease than an individual without these risk factor(s). These risk factors include, but are not limited to, genetic (i.e., hereditary) considerations (including family history and genetic markers) and environmental considerations (including exposure to environmental insults that can induce nonsense mutations (e.g., radiation, chemicals, and the like). It is understood that having only one risk factor can often indicate high risk. The clinician, as one skilled in the art, has discretion to determine whether treatment using an agent may be indicated for an individual at risk.

In one embodiment of the invention, a individual who is a candidate for therapy according to the invention is diagnosed with a cutaneous or GI disorder associated with a nonsense mutation. Diagnosis may be made on the basis of clinical symptoms, biochemical analyses of biological samples (including genetic testing to detect a defect in a gene implicated in the disorder), or any combination thereof. Diagnosis can be made by, for example, obtaining a nucleic acid sample from the subject and conducting an assay to detect the nonsense mutation in the gene implicated in the disorder suspected in the patient, or by use of a protein-based assay to detect truncated proteins that are the gene product of the nonsense mutation-containing gene implicated in the disorder.

Cutaneous disorders suitable for treatment according to the invention include those associated with a nonsense mutation. Cutaneous disorders of particular interest are those in which the affected cells are accessible by localized delivery to the skin, particularly by topical administration. Exemplary cutaneous disorders associated with nonsense mutations include, but are not necessarily limited to: basal cell nevus syndrome (e.g., PTCH gene), sporadic basal cell carcinoma (e.g., PTCH gene), melanoma (e.g., CDKN2a gene), junctional epidermolysis bullosa (e.g., LAMB3, LAMC2, LAMA3 genes), generalized atrophic benign epidermolysis bullosa (e.g., COL17A1 gene), dystrophic epidermolysis bullosa (e.g., COL7A1 gene), Hailey-Hailey disease (e.g., ATP2C1 gene), Darier's disease (e.g., ATP2A2 gene), lamellar icthyosis (e.g., TGM1 gene), X-linked icthyosis (e.g., STS gene), xeroderma pigmentosa (e.g., XPA, XPC, XPG genes), Bloom syndrome (e.g., BLM gene), striate palmo-plantar keratoderma (e.g., DSP, DSG1 genes), Cockayne syndrome (e.g., ERCC6 gene), oculocutaneous albinism (e.g., TYR, TYRP1 genes), Hermansky-Pudlack syndrome (e.g., HPS1, HPS4 genes), ataxia-telangiectasia (e.g., ATM gene), Griscelli syndrome (e.g., RAB27A, MYO5A genes), ectodermal dysplasia/skin fragility (e.g., PKP1 gene).

Gastrointestinal disorders caused by a nonsense mutation (and/or having one ore more symptoms caused by a nonsense mutation) and in which the affected cells are accessible by localized delivery to a mucosal surface of the gastrointestinal system are amenable to therapy according to the invention. Exemplary gastrointestinal disorders associated with nonsense mutations include, but are not necessarily limited to: sporadic cancers of the esophagus (p53 gene) and colon (APC, p53 genes), Barrett's esophagus (p53 gene), hereditary cancer syndromes such as adenomatous polyposis coli (APC gene), hereditary nonpolyposis colon cancer (MLH1, MSH2 genes), Peutz-Jeghers syndrome (STK 11 gene), and Cowden's syndrome (PTEN gene).

Monitoring Therapy

Therapy according to the invention can be monitored, and dosages and regimen adjusted accordingly, by assessing the effect of therapy upon one or more clinical symptoms. In general, an effective amount of a nonsense suppressor agent is a dose or doses that provide for an improvement in one or more clinical symptoms in the subject.

Where the disorder is a cutaneous or gastrointestinal disorder and the agent is an aminoglycoside, topical or intraluminal administration of the agent has the advantage of generating therapeutic concentrations locally, without resulting in toxic systemic levels. Monitoring of blood levels of aminoglycoside antibiotics is a mature technology, routinely used in hospitalized patients receiving aminoglycosides for serious bacterial infections. Methods for monitoring aminoglycoside levels are known in the art (see, e.g., U.S. Pat. No. 5,858,805). Widespread or prolonged topical or intraluminal use can be coupled with monitoring of blood levels to assess aminoglycoside levels so as to avoid onset of systemic toxicity.

In general, therapy can be monitored by assessing one or more clinical parameters. Monitoring clinical parameters can facilitate assessment of initial responsiveness to therapy and/or efficacy, as well as the appropriate dose or dosage of the therapeutic agent. Exemplary clinical parameters include, but are not necessarily limited to, severity or extent disease symptoms compared to prior to therapy or to an unaffected individual, and the like. Assessing levels of production of a full-length and/or functional gene product encoded by the gene containing the nonsense mutation which is associated with disease can also be used to monitor therapy.

It is understood that monitoring therapy means that symptoms are assessed at different times and are compared over time. Where assessment of a clinical symptom requires analysis of a biological sample, such biological sample(s) are generally obtained at different times, for example, during application of therapy, and are compared, either with each other, a control, and/or a desired value.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### In Vitro Demonstration of Efficacy

Xeroderma pigmentosum is a disease of defective DNA repair. Affected patients are extremely susceptible to ultraviolet light-induced skin cancers, and without severe restriction of exposure to natural light, develop tens to hundreds of skin cancers in the first few decades of life. Xeroderma pigmentosum complementation group A cell line XP12RO contains homozygous nonsense mutations in the XPA gene.

Such XP12RO cells are contacted with an aminoglycoside such as gentamicin to assess the effects upon promote readthrough of the relevant nonsense mutations, thereby permitting expression of functional XPA protein. Since the XPA protein is involved in repair of DNA damage caused by ultraviolet light, a rapid assay for the expression of XPA protein is survival of cells when exposed to ultraviolet light. The effects of an aminoglycoside on readthrough can also be assessed using an immunoassay for XPA protein, and to further confirm that enhanced cell survival in the presence of the aminoglycoside is due to production of XPA protein.

Hailey-Hailey disease is a chronic disease of skin fragility, caused by mutation of the ATP2C1 gene, which encodes a calcium pump. Keratinocytes from affected patients have an abrogated intracellular calcium concentration change in response to changes in extracellular calcium concentration compared to wild type cells. The intracellular response to extracellular calcium in the presence of a nonsense suppressor agent, such as the aminoglycoside gentamicin, can be monitored by use of colorimetric probes.

### Example 2

### Aminoglycosides in the Treatment of a Nonsense Mutation-Associated Cutaneous Disorder

In patients with xeroderma pigmentosum, with known nonsense mutations in causative genes, regular administration of a topical aminoglycoside to sun-exposed skin is initiated, in order to promote expression of the product of the mutated DNA repair gene. An initial dose of an about 0.3% gentamicin ointment, applied twice daily, is administered to the patient. The ultimate measure of therapeutic efficacy is based on the number of new skin cancers that the patient develops (e.g., a reduction in the number of new skin cancers in a patient receiving therapy compared to an expected number of such new skin cancers in a patient who would not receive therapy).

A more rapid assessment of efficacy can involve assessing expression of the product of the mutated XP gene in a biopsy of treated skin. In one approach, the nonsense suppressor agent is administered to non-sun exposed skin, with biopsy sampling to assess the molecular response, to assess an appropriate dosing regimen when this treatment is applied to a disease of cancer predisposition. If an adequate response, monitored either by development of new cancers or by expression of protein, is not observed, dose frequency is increased (e.g., to three or four daily applications) and/or the concentration of agent (e.g., gentamicin) is increased, with such increased being limited only by presentation of local irritation.

In patients with a blistering disease with known causative nonsense mutations, such as junctional epidermolysis bullosa or Darier's disease, topical administration of nonsense suppressor agent, such as an aminoglycoside, to sites of predilection for blistering would be initiated. For example, initial dosing can involve 0.1% gentamicin ointment, administered twice daily, due to poor barrier function in the affected areas. Therapeutic response can be monitored by decreased skin fragility in the treated areas. Increases or decreases in the frequency or strength of gentamicin administration is used to tailor the therapy based on clinical response.

### Example 3

### Aminoglycosides in the Treatment of a Nonsense Mutation-Associated GI Disorder

Several hereditary conditions that predispose to development of colon cancer are caused by nonsense mutations in known genes. For a patient with such a condition, oral treatment with a nonsense suppressor agent, such as an oral aminoglycoside, is initiated to promote readthrough of the nonsense codons and expression of the product of the mutated gene(s).

For example, paromomycin, available in oral form, is dosed to the patient at about 35-45 mg/kg daily, divided into three doses. This regimen is that suggested for treatment of intestinal amoebiasis, and thus is a reasonable beginning dose for therapy according to the invention. Determination of an optimal dosing regimen for suppression of nonsense mutations can be assessed by examining production of full-length gene products, which indicate that the agent is promoting readthrough of the mutated gene(s). Intermittent dosing may be adequate, depending on the lifetime of the expressed proteins. The adequacy of dosing in an individual patient can be monitored during routine endoscopic examinations that these patients undergo, assessing the morphology of the colonic epithelium (to assess the development of premalignant or malignant lesions) as well as obtaining biopsy specimens for determination of the presence of the product of the mutated genes.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of treating a subject having or at risk of a cutaneous disorder associated with a nonsense mutation, the method comprising:
administering locally to skin of the subject an amount of a nonsense suppressor agent effective to suppress the nonsense mutation in an affected cell in the subject.

2. The method of claim 1, wherein the nonsense suppressor agent is an aminoglycoside.

3. The method of claim 1, wherein the aminoglycoside is gentamicin.

4. The method of claim 2, wherein said aminoglycoside is streptomycin, amikacin, kanamycin, tobramycin, netilmicin, neomycin, framycetin, negamycin, paromomycin, sisomicin, or G-418, or a derivative or analog thereof.

5. The method of claim 1, wherein said administering is by topical administration.

6. The method of claim 5, wherein the agent is administered as a cream or ointment.

7. A method of treating a subject having or at risk of a gastrointestinal disorder associated with a nonsense mutation, the method comprising:
administering to an intraluminal gastrointestinal surface of the subject an amount of a nonsense suppressor agent effective to suppress the nonsense mutation in an affected cell in the subject.

8. The method of claim 7, wherein the nonsense suppressor agent is an aminoglycoside.

9. The method of claim 8, wherein the aminoglycoside is gentamicin.

10. The method of claim 8, wherein said aminoglycoside is streptomycin, amikacin, kanamycin, tobramycin, netilmicin, neomycin, framycetin, negamycin, paromomycin, sisomicin, or G-418, or a derivative or analog thereof.

11. The method of claim 9, wherein said administering is by oral administration, wherein the aminoglycoside is administered in a dose less than that effective for systemic delivery of the aminoglycoside for antimicrobial therapy.
